(19) United States
(12) Patent Application Publication
(10) Pub. No.: US 2024/0249848 A1
(43) Pub. Date: Jul. 25, 2024

(54) METHOD AND SYSTEM FOR MONITORING OF A PHYSICAL ENVIRONMENT'S PRONENESS TO INFECTIOUS DISEASE TRANSMISSION

(71) Applicant: NEC Laboratories Europe GmbH, Heidelberg (DE)

(72) Inventors: Guerkan SOLMAZ, Heidelberg (DE); Giuseppe SIRACUSANO, Heidelberg (DE); Flavio CIRILLO, Heidelberg (DE); Martin BAUER, Heidelberg (DE)

(21) Appl. No.: 18/288,828

(22) PCT Filed: May 17, 2021
(86) PCT No.: PCT/EP2021/062977

§ 371 Date: Oct. 30, 2023

(51) Int. Cl. G16H 50/80; G16H 50/20

(52) U.S. Cl. CPC G16H 50/80; G16H 50/20

(57) ABSTRACT

A method for monitoring of a proneness of a physical environment to infectious disease transmission includes a training phase in which unlabeled sensor data is obtained from sensors of the physical environment in order to provide a set of sensor features. A labeling matrix that is fed to a generative model is generated by applying situation labeling functions, wherein the generative model feeds a discriminative classifier model with probabilistic labels for the sensor features, wherein the probabilistic labels of the generative model are used for training the discriminative classifier model. A subset of the sensor features is determined based on an optimization procedure by a feature selection optimizer entity. In an operational phase, the discriminative classifier model uses the subset of sensor features for detecting predefined situations which make the physical environment prone to infectious disease transmission.

### CROSS REFERENCE TO RELATED APPLICATIONS

This application is a U.S. National Phase application under 35 U.S.C. § 371 of International Application No. PCT/EP2021/062977, filed on May 17, 2021. The International Application was published in English on Nov. 24, 2022 as WO 2022/242823 A1 under PCT Article 21(2).

The project leading to this application has received funding from the European Union's Horizon 2020 research and innovation programme under grant agreement No 871249.

### FIELD

The present invention relates to a method for monitoring of a physical environment's proneness to infectious disease transmission.

Furthermore, the present invention relates to a system for monitoring of a physical environment's proneness to infectious disease transmission.

### BACKGROUND

In recent years, preventing epidemics have been one of the biggest problems for humanity and easy transmission of COVID-19 led researchers to design systems for tracing those transmissions in indoor and outdoor environments.

Most widely used mobile systems for COVID are Bluetooth contact-tracing systems. These systems require a vast majority of people to download mobile applications and enable the Bluetooth (BT) functionality of their smartphones. Furthermore, their accuracies are bounded mainly to the BT RSSIs (Received Signal Strength Indicators) which are highly noisy due to various environmental factors. In this regard, it is exemplarily referred to the non-patent literature of G. Solmaz, J. Fürst, S. Aytac, and F.-J. Wu. “Group-In: Group Inference from Wireless Traces of Mobile Devices.” In Proceedings of ACM IEEE IPSN'20, April 2020.

Some applications of mobile systems are designed specifically for indoor systems using either people-centric sensors (sensors carried by humans) such as smartphone sensors, infrared or ultra-wide band (UWB) sensors or building sensors (sensors deployed in the building) such as CO2, wireless (WiFi/BT), acoustic, temperature, and humidity sensors. These systems mostly require costly and time-consuming ground-truth data collection campaigns for each specific environment and precise calibration for sensory data that may lead the system operating not efficiently in dynamically changing scenarios in indoor environments (e.g., seasonal changes, change in room occupancies).

Most of the existing systems work on simplistic rules such as simple distance thresholds for UWB, whereas the distance thresholds are calibrated specifically for each different device. There are some systems that are trained using more fine-grained models, signal processing (e.g., systems using WiFi sensing), or off-the-shelf supervised machine learning models (e.g., Support Vector Machine, Random Forest, Decision Tree). These systems mostly require lots of data collection and calibration for any individual device and specifically for each of the data modality or feature (e.g., WiFi signal features such as time of arrival). Other than having the cost and effort of learning these feature behaviors, these known systems are also prone to errors when they are deployed in different indoor environments.

### SUMMARY

In an embodiment, the present disclosure provides a method for monitoring of a proneness of a physical environment to infectious disease transmission. The method comprises in a training phase: obtaining unlabeled sensor data from sensors of the physical environment in order to provide a set of sensor features; generating, by applying situation labeling functions, a labeling matrix that is fed to a generative model, wherein the generative model feeds a discriminative classifier model with probabilistic labels for the sensor features, wherein the probabilistic labels of the generative model are used for training the discriminative classifier model; determining, by a feature selection optimizer entity, a subset of the sensor features based on an optimization procedure; and in an operational phase: using, by the discriminative classifier model, the subset of sensor features for detecting predefined situations which make the physical environment prone to infectious disease transmission.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 is a schematic view illustrating a data programming approach for zero hand-label training;

FIG. 2 is a schematic view illustrating a method and a system in accordance with an embodiment of the present invention;

FIG. 3 is a schematic view illustrating a method and a system in accordance with an embodiment of the present invention;

FIG. 4 is a code example illustrating exemplary sensor feature predicates for a method and a system in accordance with an embodiment of the present invention;

FIG. 5 is a code example illustrating an exemplary feature cost vector for a method and a system in accordance with an embodiment of the present invention;

FIG. 6 is a code example illustrating graph generation based on an exemplary feature matching vector for a method and a system in accordance with an embodiment of the present invention;

FIG. 7 is a code example illustrating an exemplary situation labeling threshold for a method and a system in accordance with an embodiment of the present invention;

FIG. 8 is a code example illustrating an exemplary labeling function for a method and a system in accordance with an embodiment of the present invention;

FIG. 9 is an exemplary labeling matrix for a method and a system in accordance with an embodiment of the present invention;

FIG. 10 is a schematic view illustrating an exemplary neural network model as discriminative machine learning classifier model in accordance with an embodiment of the present invention;

FIG. 11 is a listing illustrating an optimization for feature selection for a method and a system in accordance with an embodiment of the present invention;

FIG. 12 is a schematic view illustrating an implementation of a system for monitoring a physical environment's proneness to infectious disease transmission in accordance with an embodiment of the present invention; and

FIG. 13 is a schematic view illustrating a further implementation of a system for monitoring a physical environment's proneness to infectious disease transmission in accordance with an embodiment of the present invention.

### DETAILED DESCRIPTION

In accordance with an embodiment, the present invention improves and further develops a method and a system of the initially described type for monitoring of a physical environment's proneness to infectious disease transmission in such a way that an efficient monitoring is achieved.

In accordance with an embodiment, the present invention provides a method for monitoring of a physical environment's proneness to infectious disease transmission, the method comprising:
in a training phase:
   obtaining unlabeled sensor data from sensors of the physical environment in order to provide a set of sensor features;
   generating, in particular by applying situation labeling functions, a labeling matrix that is fed to a generative model, wherein said generative model feeds a discriminative classifier model with probabilistic labels for the sensor features, wherein the probabilistic labels of the generative model are used for training the discriminative classifier model;
   determining, in particular by a feature selection optimizer entity, a subset of the sensor features based on an optimization procedure; and
in an operational phase:
   using, by the discriminative classifier model, the subset of sensor features for detecting predefined situations, which make the physical environment prone to infectious disease transmission.

Furthermore, in accordance with an embodiment, the present invention provides a system for monitoring of a physical environment's proneness to infectious disease transmission, the system comprising a functional unit having one or more computational processors with access to memory, which, alone or in combination, are configured to provide for execution of the following steps:
in a training phase:
   obtaining unlabeled sensor data from sensors of the physical environment in order to provide a set of sensor features;
   generating, in particular by applying situation labeling functions, a labeling matrix that is fed to a generative model, wherein said generative model feeds a discriminative classifier model with probabilistic labels for the sensor features, wherein the probabilistic labels of the generative model are used for training the discriminative classifier model;
   determining, in particular by a feature selection optimizer entity, a subset of the sensor features based on an optimization function; and
in an operational phase:
   using, by the discriminative classifier model, the subset of sensor features for detecting predefined situations, which make the physical environment prone to infectious disease transmission.

Finally, in accordance with an embodiment, the present invention provides a non-transitory, computer-readable storage medium having instructions thereon which, upon execution on one or more processors, provide for execution of the following steps:
in a training phase:
   obtaining unlabeled sensor data from sensors of the physical environment in order to provide a set of sensor features;
   generating, in particular by applying situation labeling functions, a labeling matrix that is fed to a generative model, wherein said generative model feeds a discriminative classifier model with probabilistic labels for the sensor features, wherein the probabilistic labels of the generative model are used for training the discriminative classifier model;
   determining, in particular by a feature selection optimizer entity, a subset of the sensor features based on an optimization function; and
in an operational phase:
   using, by the discriminative classifier model, the subset of sensor features for detecting predefined situations, which make the physical environment prone to infectious disease transmission.

Embodiments of the present invention propose a solution that enables monitoring of a physical environment's proneness to infectious disease transmission (e.g., COVID-19, common cold). In particular, according to embodiments of the invention the proposed solution can leverage sensor data and domain knowledge, preferably through a novel machine learning method, which enables high-accuracy, scalable, and privacy-aware monitoring of the physical environment, in particular a target indoor environment. Thus, for instance, the physical environment may be an indoor environment.

According to the invention, it has first been recognized that an efficient monitoring is achieved by obtaining unlabeled sensor data from sensors of the physical environment in order to provide a set of sensor features and generating, in particular by applying situation labeling functions, a labeling matrix that is fed to a generative model. The generative model feeds a discriminative classifier model with probabilistic labels/predictions for the sensor features, wherein the probabilistic labels/predictions of the generative model are used for training the discriminative classifier model. Then, based on an optimization procedure, a subset of the sensor features is determined, in particular by a feature selection optimizer entity. This is performed in a training phase. Then, in an operational phase, the discriminative classifier model uses the subset of sensor features for detecting predefined situations, which make the physical environment prone to infectious disease transmission. Thus, an efficient monitoring can be achieved.

The term “feature selection optimizer entity” may be understood, in particular in the claims, preferably in the description as a software functionality. The feature selection optimizer entity might be implemented as being part of a computer system, which implements an algorithm for performing an optimization procedure for selecting an suitable subset of sensor features.

According to embodiments, the proposed solution may enable monitoring environments' proneness to transmission of infectious diseases. The solution may constantly monitor and/or detect the predefined situations, which make the environment prone to the transmissions. Embodiments may provide a novel way on how to train these situations without vast ground-truth data collection and calibration efforts and can provide best performance based on the data availability and the constraints in the physical environments. Data availability may involve availability of sensory data and domain knowledge, whereas constraints may involve deployment costs such as energy costs, privacy concerns, or other rules/regulations imposed on social environments.

Embodiments of the invention may fall into the category of data programming (cf. the non-patent literature of Ratner, Alexander, et al. “Data programming: Creating large training sets, quickly.” Advances in neural information processing systems 29 (2016): 3567) and feature selection algorithms, whereas they may also leverage supervised machine learning models such as Logistic Regression (Log R), Random Forest (RF) and/or Artificial Neural Network (ANN) models.

According to embodiments of the invention, it may be provided that, in the training phase, feature cost information for the sensor features are received from a knowledge base, wherein the feature cost information is used for considering a predetermined constraint metric for the physical environment. The feature cost information may be handled in a feature cost vector, wherein the feature cost vector is taken from the knowledge base based on a predetermined cost metric.

According to embodiments of the invention, it may be provided that, in the training phase, sensor feature predicates are received from a knowledge base, wherein the sensor feature predicates indicate/specify characteristics of the sensor features. Thus, the sensor feature predicates can be are taken from the knowledge base based on characteristics of the sensory data features.

According to embodiments of the invention, it may be provided that a match score is employed for considering a level of matches of sensor feature predicates between a pair of sensor features. The match score between every pair of sensor features may be handled through one to one matching between their predicates. For instance, an implementation of the matching procedure could the matching starting from <predicate_0> to <predicate_n> and when there exists a mismatch, the algorithm does not continue the matching procedure. This can be a simple implementation choice. Furthermore, the match score between every sensor feature pair may be handled through the one to one matching between their predicates. Then, it may be provided that in the case of there is any match between any pair of predicates occur at the same level or order (i.e., when <predicate_i> of feature fa is identical to the <predicate_i> of feature fb), the two features are considered “connected features”. This information would be useful in the later step of automatic feature graph generation. A correct match for any predicate <predicate_i> may add a value m; to the match score. The total sum of the matchings could be considered the match score between the two features. These match scores may be used for generating a graph, where the features are represented by the vertices, connection between features are represented by edges and the match scores are represented by edge weights.

According to embodiments of the invention, it may be provided that, in the training phase, a feature node dependency graph is generated based on the sensor feature predicates of the sensor features, wherein a match between the sensor feature predicates of two sensor features constitutes a dependency between the two sensor features. Thus, dependencies between pairs of sensor features can be considered, in particular such that the feature node dependency graph indicates/specifies how much similarity exits between a pair of sensor features.

According to an embodiment, the feature node dependency graph may be generated in such a way that the sensor features are represented by vertices, connections between sensor features are represented by edges, and match scores are represented by edge weights.

According to embodiments of the invention, the optimization procedure performed by the feature selection optimizer entity may be based on a traversal of the feature node dependency graph. Furthermore, it may be provided that the optimization procedure performed by the feature selection optimizer entity is based on feedbacks received from the discriminative classifier model.

According to embodiments of the invention, the optimization procedure may include an optimization function, wherein the optimization function is built based on the feature node dependency graph, in particular based on the edge weight values.

According to embodiments of the invention, the optimization procedure may include an optimization function, wherein said optimization function is built based on a feature cost vector, wherein said feature cost vector includes feature cost information for the sensor features.

According to embodiments of the invention, the optimization procedure may include an optimization function, wherein the optimization function is built based on training and/or prediction times of the generative model and/or of the discriminative classifier model.

According to embodiments of the invention, the optimization procedure may include an optimization function, wherein the optimization function is built based on prediction accuracy and/or confidence values of the discriminative classifier model.

According to embodiments of the invention, it may be provided that, in the training phase, the feature selection optimizer entity iteratively interacts with the discriminative classifier model in order to find a less costly subset of features to be used for the operational phase. For instance, in the training phase, the feature selection optimizer entity may iteratively interact with the discriminative classifier model such that the subset of sensor features is iteratively updated based on feedback information that is provided by the discriminative classifier model, and wherein the discriminative classifier model is trained on the probabilistic labels. Thus, a subset of the sensor features with minimal cumulative feature costs can be selected and training the discriminative classifier model may be trained by computing a loss function (as optimization function) until convergence.

According to embodiments of the invention, the probabilistic labels/predictions of the generative model may be based on the unlabeled sensor data and predetermined situation labeling thresholds. Thus, the situation labeling thresholds can be used as instantiation parameters for the situation labeling functions.

According to embodiments of the invention, a dynamic feature programming and data programming may be provided, which quantifies knowledge of domain experts using knowledge base to set situation thresholds and cost vectors for given indoor environments. Embodiments may provide a way of leveraging these inputs for accurate, scalable, and cost efficient environments' proneness monitoring for disease transmission: Generation and traversal of a graph of feature nodes automatically based on the given feature predicates and associated feature vectors and feature learning objective optimization through feedback from data programming through situation labeling functions.

According to embodiments of the invention, a knowledge base may provide situation labeling functions, situation labeling thresholds, feature costs and sensor feature predicates. For example, a knowledge base may report the cost of using video based features coming from camera with very high costs (due to privacy, bandwidth consumption, or processing computation).

According to embodiments of the invention, data programming may be used to identify a minimum set of features needed for the performance of accurate and cost-efficient disease transmission monitoring.

According to embodiments of the invention, the output of the (final) discriminative classification model may infer the proneness of an indoor environment (how healthy is the air-salubrity) and it might be connected to the HVAC (Heating, ventilation, and air conditioning) system for activating ventilation or opening windows.

Embodiments of the present invention may have one or more of following advantages:
It may be provided that embodiments does not require ground-truth data for monitoring disease transmission.
Embodiments may automatically adapt to differences between different environments/scenarios and situations.
Embodiments may provide a unique way of bringing domain knowledge into the machine learning through feature programming and data programming.
Embodiments can be useful in environments such as airports, hospitals, business areas, or governmental buildings. For example, embodiments can be helpful to end national lockdowns and opening up schools.

There are several ways how to design and further develop the teaching of the present invention in an advantageous way. To this end it is to be referred to the dependent claims on the one hand and to the following explanation of further embodiments of the invention by way of example, illustrated by the figure on the other hand. In connection with the explanation of the further embodiments of the invention by the aid of the figure, generally further embodiments and further developments of the teaching will be explained.

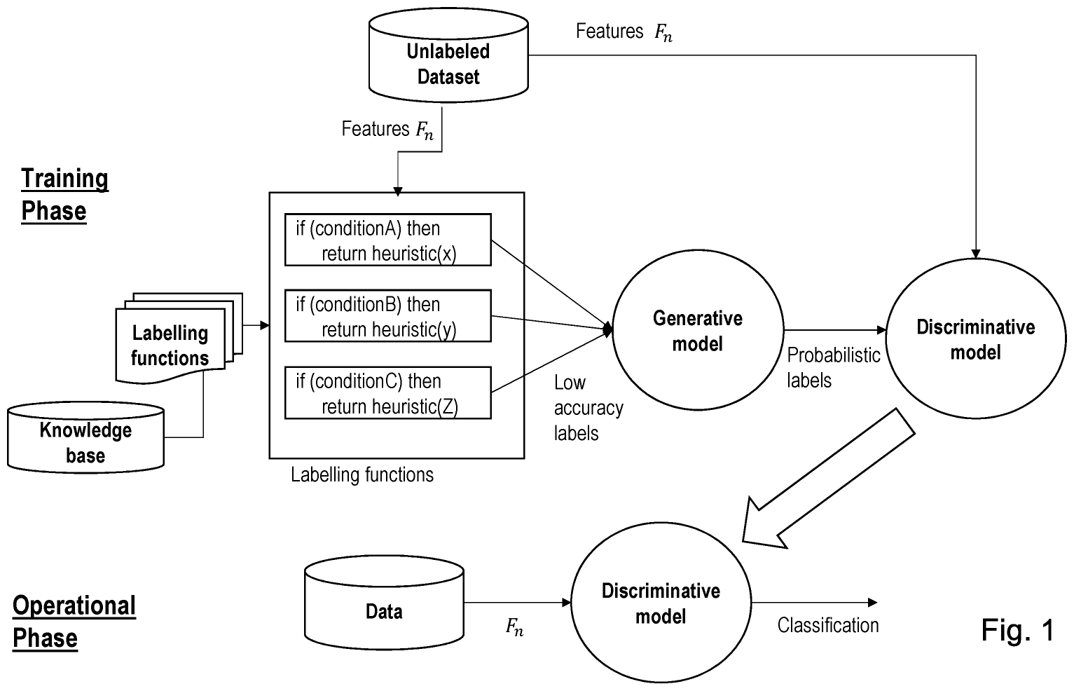

FIG. 1 shows a data programming approach for zero hand-label training. This approach is based on using functions to programmatically label data as opposed to hand-label each data point. Data programming may be an approach for training a system without ground-truth data, wherein a knowledge base provides different labeling functions that are heuristics for labeling data points with low accurate and low coverage. Once applied to an unlabeled dataset, each labeling function computes a label for each data point of the dataset. Different labeling functions might produce contrasting labels for the same data points. A labeling function might also return abstain if the conditions of the heuristic are not matched.

Applying the labeling functions to a training dataset generates a matrix that is fed to a generative model. The generative model decides for each data point a single label. A very simplistic approach might be to apply a majority voter to decide the final labels. Approaches that are more sophisticated may envision the usage of probabilistic means.

The training dataset together with the (probabilistic) labels generated by the generative model are used to train a discriminative model. Once trained, the discriminative model is used at an operational phase to make classifications on other data.

The above design assumes that the set of n features Fₙ used to train the discriminative classifier model is decided by a domain expert. However, it might happen that the features are costly from different point of views, such as computation (e.g., object detection on video stream) or privacy (e.g., MAC address of a Bluetooth device). For example, in the privacy case, a system developer would prefer to avoid the usage of any personal data in order to avoid any infringement of GDPR (General Data Protection Regulation) or starting a legal procedure to use personal data (i.e., asking consent to each individuals). The GDPR is hard to be solved at the operational phase, since it might be an online phase, such as the disease spread monitoring. However, choose the best set of features to have good classification accuracy and minimizing the features' costs is not an easy task.

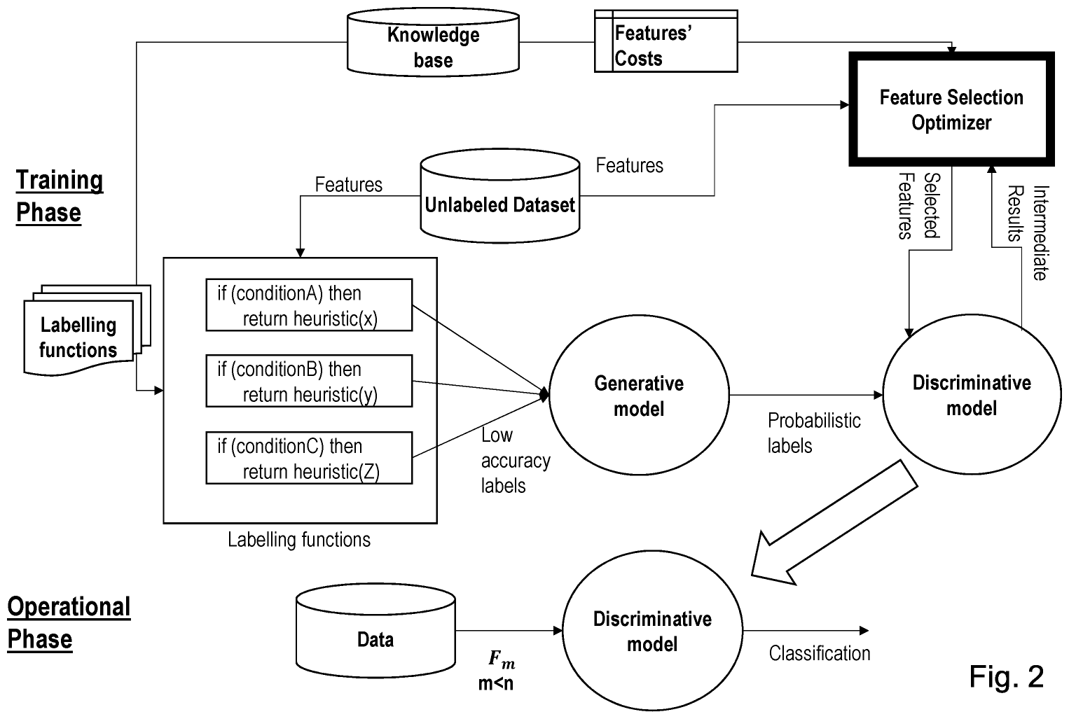

FIG. 2 shows method and a system in accordance with an embodiment of the present invention. The embodiment proposes a method and a system that enable monitoring of (physical) environments' proneness to infectious disease transmission (e.g., COVID-19, common cold). The embodiment may leverage IoT (Internet of Things) sensors as well as domain knowledge through a novel procedure, which enables high-accuracy, scalable, and privacy-aware monitoring of a target indoor environment. This procedure may be described as “dynamic feature programming and data programming”.

The embodiment considers target environment's proneness to be dynamically changing by various environmental factors, movement of people, and other dynamics such as connection to other environments. Compared to known state-of-the-art systems, embodiments of the invention can provide an easily programmable, quantifiable, and cost-efficient way without extensive data collection and calibration efforts. Thus, the applicability of disease transmission monitoring in indoor environments can be improved.

Main technical benefits of embodiments in accordance with the present invention may be to remove the cost of ground-truth data collection and excessive calibration for indoor mobile systems by leveraging domain knowledge through knowledge bases for sensor data features and data labels. More specifically, technical benefits of embodiments of the invention may include:
Reducing development and deployment costs,
Providing a privacy-by-design way for non-invasive monitoring, and
Automatically adapting scenarios based on availability of IoT devices (e.g., sensors) and constraints of these scenarios.

Embodiments of the invention address the above-mentioned issues by introducing a component into the data programming approach at training phase (cf. FIG. 2), namely the feature selection optimizer, which takes as input the sensor features and a cost vector for the sensor features and selects an optimal set of features for the targeted classifier's performance. The feature selection optimizer iteratively interacts with the discriminative model to find the less costly set of sensor features. The final discriminative model will expect a set of m features Fₘ with m<n.

An embodiment of the invention leverages various devices such as IoT devices for raw data collection (data without ground-truths) and machine learning for monitoring and detection the predefined situations that may lead to spread of diseases in the indoor environments. Unlike most known state-of-the-art systems, the embodiment of the invention does not aim to trace individuals for their contacts to the people who allegedly have the disease. Instead, it aims to monitor the environmental dynamics, which might lead to environments' proneness to the disease transmissions. This main difference enables a method and a system in accordance with the invention to successfully operate without identifying people in the environment through unique IDs, face recognition or others.

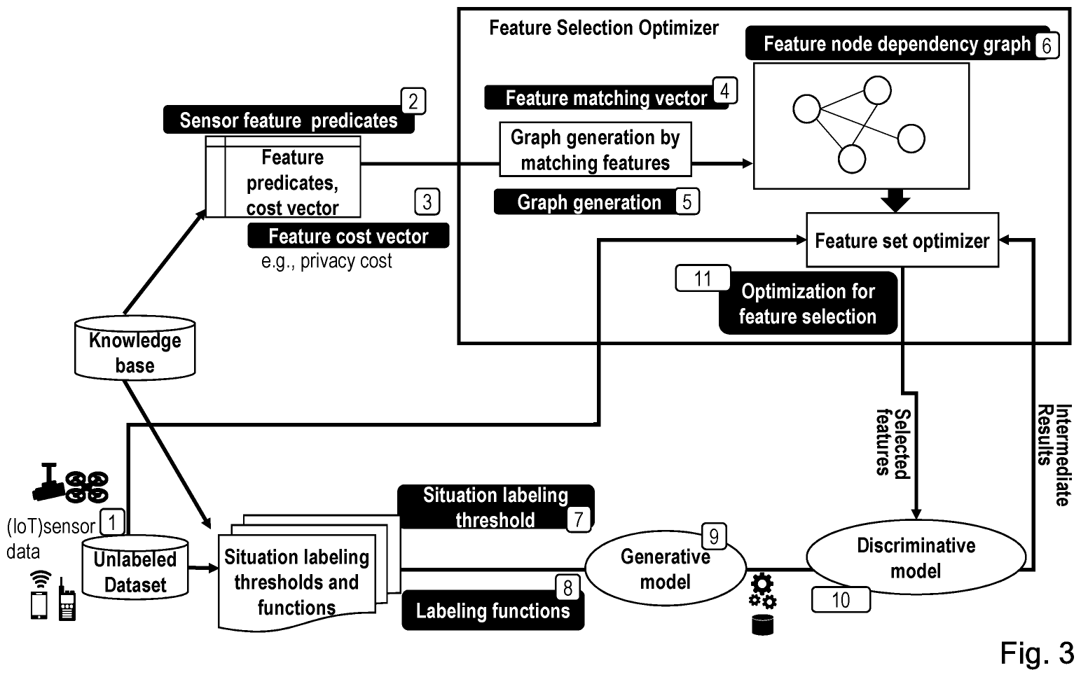

FIG. 3 shows a method and a system in accordance with an embodiment of the present invention. The embodiment provides an environmental monitoring and enables feature programming for feature learning and data programming for weak supervision.

The embodiment may require domain knowledge from a “knowledge base”, as illustrated in FIG. 3. The knowledge base may provide the following inputs to a system in accordance with an embodiment of the invention:
sensor feature predicates,
feature cost vectors,
situation labeling thresholds and
abstract situation labeling functions.

Through the proposed solution in accordance with an embodiment of the invention, unlabeled data collected from the wild can be leveraged for accurate environment monitoring for disease transmissions. Moreover, the embodiment enables a cost-efficient and privacy-aware system design. Thus, it is available for use in real scenarios more flexibly, compared to known state-of-the-art systems and methods.

A method in accordance with an embodiment of the invention may include the following steps. These steps are mapped to FIG. 3 with numbers inside rectangles.

Step 1 (cf. FIG. 3): (IoT) sensor data collection from various devices connected with a set of sensors is performed. The devices and sensors are considered to be heterogeneous in their nature and they may produce noisy and sparse measurements. Possible devices are previously listed as possible system components. The data collected from the (IOT) sensors such as image data (e.g., from drones or cameras), or wireless data (e.g., from WiFi scanners or mobile devices such as smartphones) are considered as the “raw” data without the ground-truth labels associated with them. For instance, for the case of the environmental monitoring, a ground-truth might be the times of social interactions that might lead to disease transmission.

The collected (IoT) sensor data will be processed through various steps that are described below.

Step 2 (cf. FIG. 3): Sensor feature predicates are taken from the knowledge base based on the characteristics of the sensory data features. Every feature may or may not have multiple predicates in front of it, followed by a name of the feature. The predicates may be written in the following format:

<predicate_0>_<predicate_1>_..._<predicate_k>_<feature_index>_<feature_identifier>,
where 0≤k, k ∈ Z⁺.

The predicates are listed from a more general to a more specific order. For instance, if two sensor features have commonality in a general level (e.g., both sensors are wireless), than this information can be coded as the initial predicate (i.e., such as <predicate_0>=“wireless”). The more specific features such as “bluetooth” or “wifi” might be entered as the second or later predicates (i.e., such as <predicate_1>=“bluetooth” or <predicate_1>=“wifi”). Below are some example feature names along with the feature predicates that are entered to the knowledge base.

Feature: wireless_rssi_wifi
Feature: wireless_rssi_bluetooth
Feature: room_presence_illuminance
Feature: room_presence_pir
Feature: room_climate_co2
Feature: room_climate_humidity
Feature: room_climate_temperature
Feature: room_noise (feature with only 1 predicate)

All feature predicates may be received from the knowledge base through a simple function call with the old name of the feature and the new name of the feature with predicates and the identifier.

Every pair of features taken from the knowledge base are used by the system for creating the one-to-one matchings of the set of predicates of each vector. For instance, wireless_rssi_wifi and wireless_rssi_bluetooth have two predicates that match with each other, which are “wireless” and “rssi”, whereas room_presence_illuminance and room_climate_humidity have only one matching predicate “room”. As expected, there is no match between two features such as wireless_rssi_bluetooth and room_noise.

For the sake of simplicity, the set of all features (n features) can be notated as a vector of features as \vec{F} as follows.

$$\vec{F} = [ {f}_{1} , {f}_{2} ... , {f}_{n} ] , n {ϵℤ}^{+} ,$$

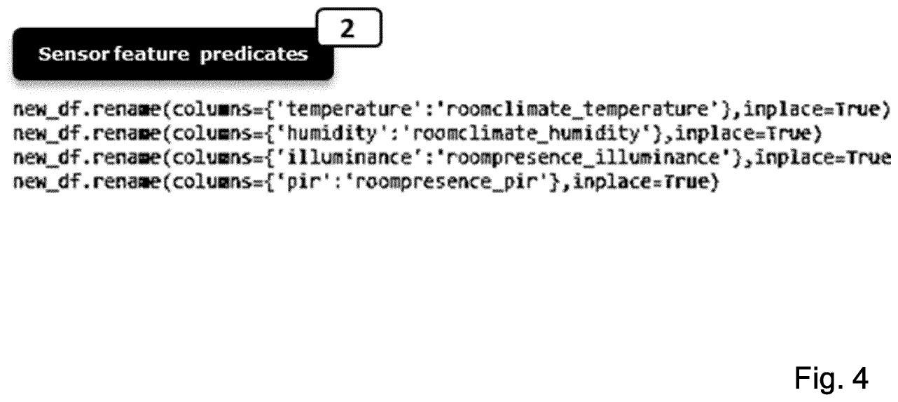

FIG. 4 shows a code example illustrating exemplary sensor feature predicates for a method and a system in accordance with an embodiment of the present invention.

Step 3 (cf. FIG. 3): Feature cost vectors are taken from the knowledge base based on the cost metric. The feature cost may include energy consumption or privacy concerns. Each element of a vector correspond to a data feature which is predefined in the previous step. Below is a definition of a cost vector \vec{C}.

$$\vec{C} = [ {c}_{1} , {c}_{2} ... , {c}_{n} ] , n {ϵℤ}^{+} , {C}_{min} \leq {c}_{i} \leq {C}_{max} ϵ {R}^{+} ,$$

where n is the number of features and cᵢ is the associated cost value of each sensor data feature. The feature cost values should be entered in the given range. For instance, the range can be [Cₘᵢₙ, Cₘₐₓ]=[0, 10] and cϵ [symbol] for a privacy cost of a feature, whereas it may be [Cₘᵢₙ, Cₘₐₓ]=[0, 200 W] and cϵ [symbol] for the energy consumption cost. The system may have an API (application programming interface) interface, which allows domain experts to enter the associated cost value of each feature to the knowledge base. The cost vector can be used at the later steps such as in the definition of the loss function and the optimization of the system.

Thus, this step allows using the knowledge base to specify feature predicates with associated costs. For example, assume there exist 2 features f₁ and f₂ with the respective feature predicates camera_image_frame and room_climate_humidity, the associated costs c₁ and c₂ can be set as {c₁=7, c₂:=0} (0≤cᵢ≤10), respectively. For example, considering a privacy constraint in the environment, this would enable penalizing the use of image frames from the camera sensor that might be privacy-sensitive whereas it would not cause any penalty later for the use of humidity sensor.

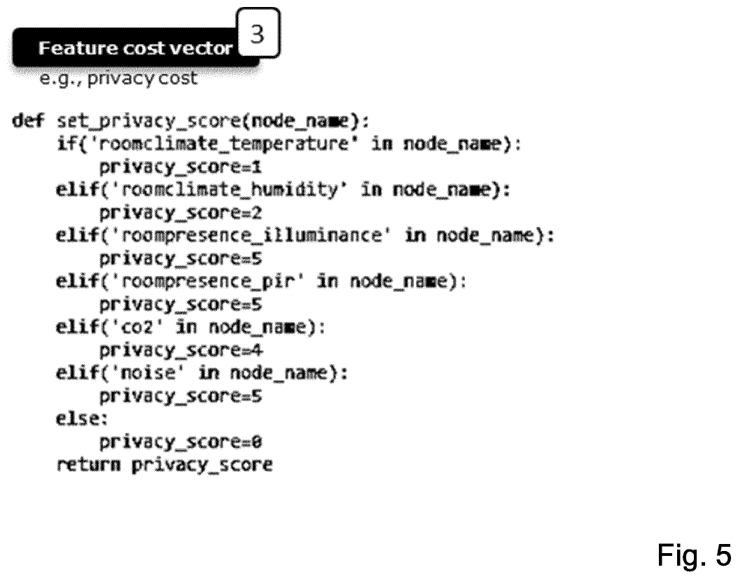

FIG. 5 shows a code example illustrating an exemplary feature cost vector for a method and a system in accordance with an embodiment of the present invention, wherein the example considers privacy costs.

Step 4 (cf. FIG. 3): Feature predicate matching vectors can be set by default by the system in accordance with an embodiment of the invention with a set of predefined values. On the other hand, these values can be easily adjusted by domain expert (similar to the above step) in the knowledge base, so to change the default matching vector of the system and define similarities/correlations between all sensor features. These features are previously defined or renamed for the knowledge base. By adjusting this vector, the knowledge base can give the system a sense of how much similarity may exist between any feature pairs.

The feature vector \vec{M} may be defined as follows.

$$\vec{M} = [ {m}_{0} , {m}_{1} ... , {m}_{n - 1} ] , n {ϵℤ}^{+} , {m}_{i + 1} \leq {m}_{i} ϵ {R}^{+} ,$$

where each matching has an associated match score value mᵢ which is a positive number. Due to the previously described sensor feature predicate convention, it is expected (as shown in the above notation) that when the match happens early-on, the value of the match would be higher than it happens later. However, in practice any match value can be given without following a particular order.

Moreover, an exemplary implementation of the matching procedure does the matching starting from <predicate_0> to <predicate_n> and when there exists a mismatch, the algorithm does not continue the matching procedure. This may be a simple implementation choice.

The match score between all sensor feature pairs are handled through one to one matching between their predicates. In the case of there is any match between any pair of predicates occur at the same level or order (i.e., when <predicate_i> of feature fₐ is identical to the <predicate_i> of feature f_{b}), then the two features are considered “connected features”. This information would be useful in the later step of automatic feature graph generation.

A correct match for any predicate <predicate_i> adds the value mᵢ to the match score. The total sum of the matchings may be considered as the match score between the two features. These match scores can be used at a later step for generating a graph, where the features are represented by the vertices, connection between features are represented by edges and the match scores are represented by edge weights.

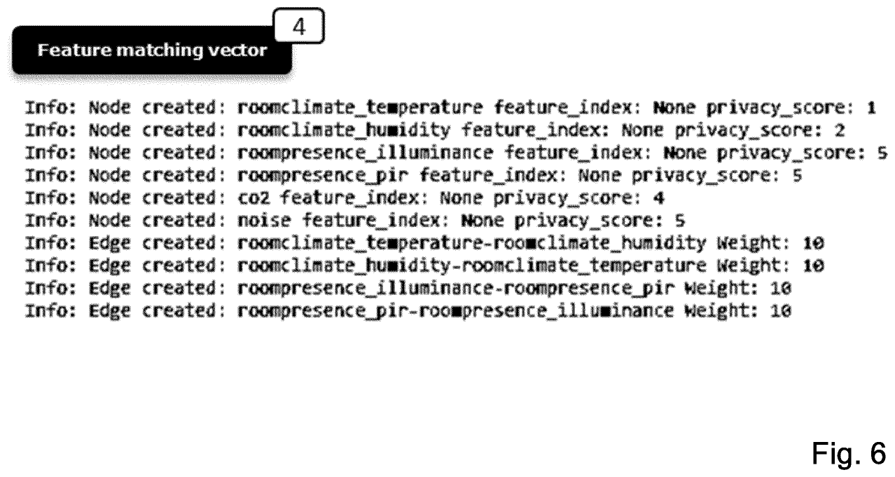

FIG. 6 shows a code example illustrating graph generation based on an exemplary feature matching vector for a method and a system in accordance with an embodiment of the present invention.

Step 5 (cf. FIG. 3): Feature graph generation is an automatic process provided by the system. The generation process is built upon the feature predicates that were entered from the knowledge base in the earlier step. The embodiment generates a graph G=(V,E), where vertices V={f₁, f₂, f₃, . . . fₙ} represents all of the possible sensory data features (n features) that the system may have (i.e., all data streams based on different data types). In G, the edges E represent the dependencies between different data features such that the weight wᵢⱼ of each edge eᵢⱼ represents the dependency value between the nodes (features) i and j, 0≤i, j≤n, i, jϵZ⁺.

Step 6 (cf. FIG. 3): Feature node dependency graph: In this step, the edge weight values wᵢⱼ for every feature pair (fᵢ, fⱼ) may be adjusted. The edge weight values may be by default set using the feature matching vector and the “match scores” between every feature pairs (fᵢ, fⱼ). The decision on the match scores is described in the earlier step. A simple example for default edge weights are shown below.

Example connection of features: Assume m₀:=2 and m₁:=1 and

$${f}_{1} {=}^{‶} {air\_co2}^{″} ,$$

$${f}_{2} {=}^{‶} {air\_humidity}^{″} ,$$

$${f}_{3} {=}^{‶} wireless\_rssi {\_wifi}^{″} ,$$

$${f}_{4} {=}^{‶} wireless\_rssi {\_bluetooth}^{″} .$$

The resulting graph G will have 4 vertices V:={f₁, f₂, f₃, f₄} and two edges with weights
w₁₂:=2->one predicate match (“air”); leading to m₀ as the edge weight
w₃₄:=3->two predicate matches (“wireless” and “rssi”); leading to addition of mᵢ and m₀

The edge weight wᵢⱼ represents the similarity of the two features. wᵢ may be by default considered as the match scores, whereas it can be easily adjusted using different heuristic algorithms or rule-based systems. Various methods can be used for setting the edge weights for ranking or quantifying the similarity/dependency between the features. Thus, the generated graph is a “feature node dependency graph” as illustrated in FIG. 3 with number 6.

Step 7 (cf. FIG. 3): Situation labeling thresholds are parameter values, which can be filled from the knowledge base. The thresholds values can be set based on the environments' needs and domain knowledge about the environment.

According to an embodiment, the situation labeling thresholds may be used as the instantiation parameters for the abstract situation labeling functions in the later step. Every abstract situation labeling function may have one or multiple situation labeling thresholds. Moreover, different threshold values can be taken from the knowledge base for the same abstract situation labeling functions and, after the threshold values are set (the situation labeling functions are instantiated) and the raw IoT sensor data start streaming, the probabilistic labels will be assigned by the situation labeling functions that provide different signals as the weak supervision sources.

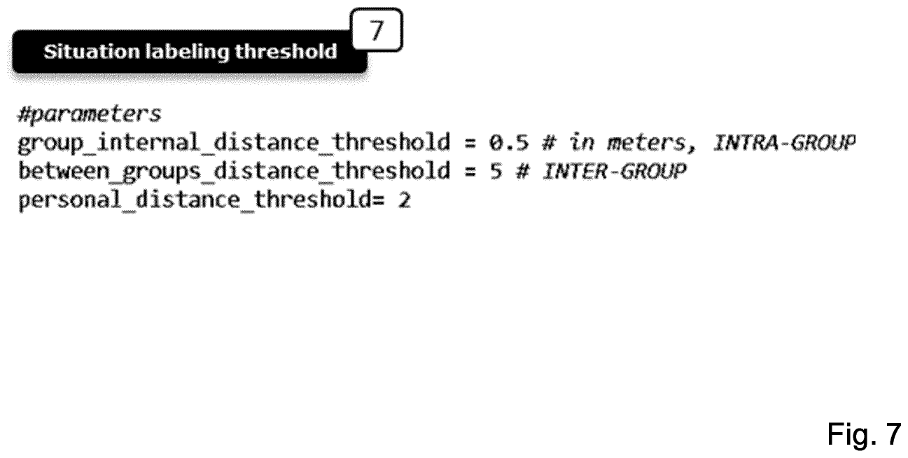

FIG. 7 shows a code example illustrating an exemplary situation labeling threshold for a method and a system in accordance with an embodiment of the present invention.

Step 8 (cf. FIG. 3): Abstract situation labeling functions are small code snippets that are abstract functions, which can be taken from the knowledge base. These functions can simply make probabilistic predictions on a possible proneness for disease transmission in the environment. To make this prediction, they may require two things: 1) (IoT) sensor data and 2) situation labeling thresholds (described in the earlier step).

Each situation labelling function may contain one or multiple sensory feature parameters as well as instantiation parameters to be used for predictions. The situation labelling functions can be used as template labeling functions and whenever the threshold values are received, they are instantiated as labelling functions. The outlook of an instantiated labeling function is already defined by the Snorkel system. In this regard, it is referred to the non-patent literature of Ratner, Alexander, et al. “Data programming: Creating large training sets, quickly.” Advances in neural information processing systems 29 (2016): 3567. An abstracted version of the programming interface such as the one suggested in Snorkel can be leveraged for entering situation labelling functions. The only difference of the interface is the difference between abstracted labeling function that are not instantiated before the threshold values are taken from the knowledge base.

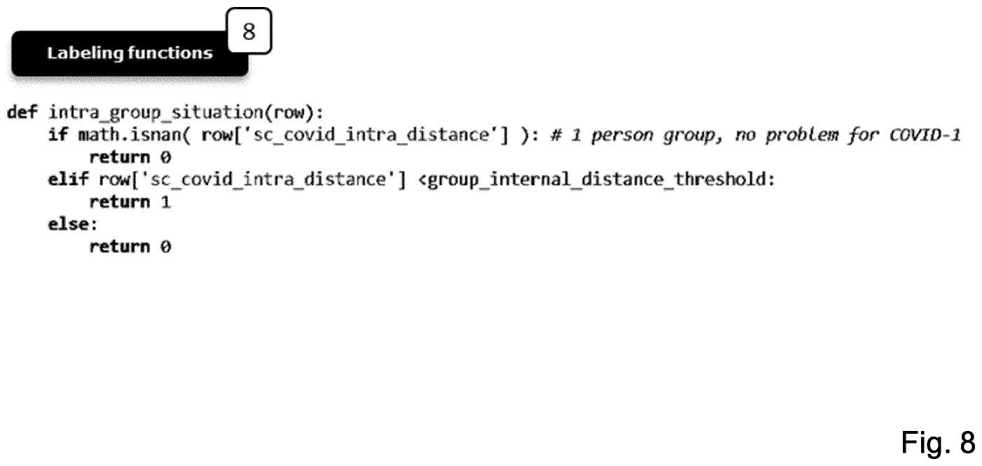

FIG. 8 shows a code example illustrating an exemplary labeling function for a method and a system in accordance with an embodiment of the present invention.

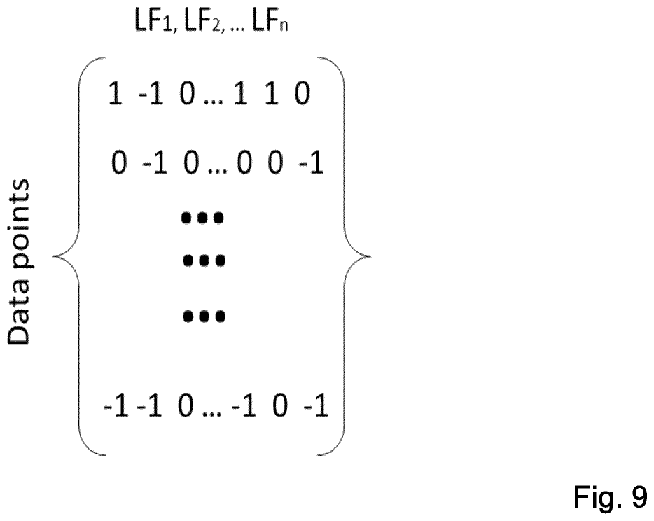

Step 9 (cf. FIG. 3): Generative models can be used for weak supervision and probabilistic predictions/labels of possible disease transmissions. The generative model uses the signals that are combined in a “labelling matrix” from the previous step for learning the structures. FIG. 9 illustrates a labelling matrix. Specifically, FIG. 9 shows a labeling matrix including the values predicted by n situation labelling functions (LFs) on each unlabeled raw data point. The values of the matrix of FIG. 9 are illustrated as binary predictions for simplicity, whereas −1 represents no output from an LF for the given data point. In practice, they may be any integer or real numbers. Various methods to explore the dependencies between situation labels can be considered for the generative model such as the majority voting or clustering methods.

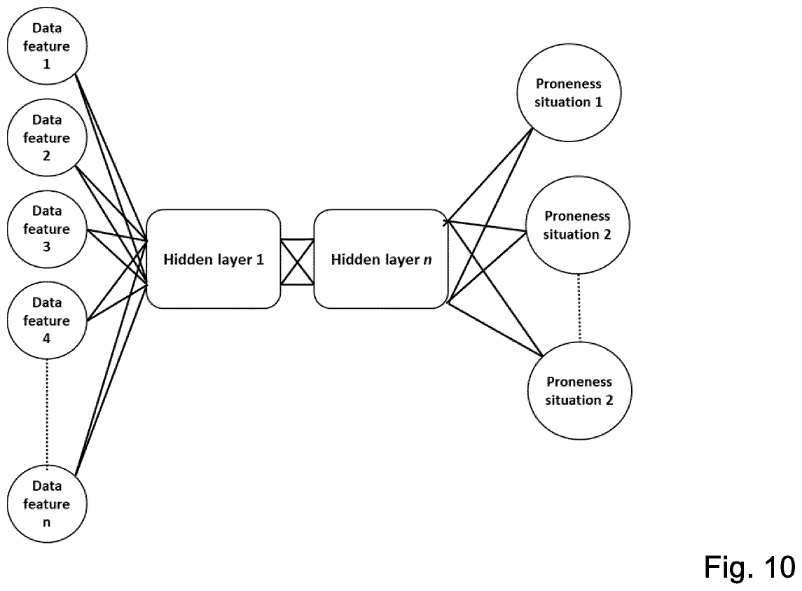

Step 10 (cf. FIG. 3): Discriminative model: The outputs of the generative model may then fed to a discriminative (machine learning) classifier model that would do the final prediction on the raw IoT sensor data, which might or might not labeled by the generative model. The main benefit of the discriminative classifier model is the generalization to a larger dataset compared to the generative model, which is bounded by the coverage of the labeling functions (e.g., as described in the non-patent literature of Ratner, Alexander, et al. “Data programming: Creating large training sets, quickly.” Advances in neural information processing systems 29 (2016): 3567). For example, a neural network model may be leveraged as the end-classifier. FIG. 10 shows a neural network model as discriminative machine learning classifier model in accordance with an embodiment of the present invention.

Step 11 (cf. FIG. 3): Optimization for feature selection in accordance with an embodiment of the invention is based on the traversal of the feature node dependency graph and feedbacks received from the discriminative classifier model. The optimization loss function may be built based on one or more of the following characteristics:
Feature cost vector \vec{C}
Training and prediction time of the generative and discriminative machine learning models
Feature node dependency graph; in particular the edge weight values
Prediction accuracy or confidence values

The optimization function penalizes addition of new sensor features in the case that they have a positive cost value in the corresponding index of the feature cost vector. As a very simple example, a complete image frame as a feature may correspond to a higher cost than a BT RSSI measurement feature. These values were previously set by the knowledge base. Furthermore, it penalizes the time consumed for training and prediction of the machine learning models. Lastly, it penalizes either prediction accuracy or confidence values (or both accuracy and confidence) based on the result of the discriminative model.

The optimization function may be updated through small batch(es) of data streams that flow through the machine learning models and the results and time spent are given as feedback information (as illustrated in FIG. 3). The considered objective optimization function may be as follows:

$$min L$$

$${L}_{b} ( {x}_{1} , {x}_{2} , ... , {x}_{m} ) = \frac{1}{m} {\sum }_{j = 1}^{m} [ α * ( {\vec{C} ( \vec{F} )}^{T} ) + β * ( ( {t}_{lab} + {t}_{gen} ) + {t}_{dis} + {t}_{pred} ) + γ * ( 1 - ρ ) ]$$

$$0 \leq ρ \leq 1 , ρ ∈ R$$

where α,β,γ are empirical parameters for adjusting the importance of feature costs, training and prediction times, and the accuracy or confidence respectively. The training and prediction times may include time for application of labeling using labeling functions t_{lab}, generative model training time t_{gen}, discriminative model training time t_{dis}, and prediction time for final labeling t_{pred}. \vec{c} represents the feature cost vector (i.e., a vector of values in a given range which quantifies the cost, e.g., cost of privacy of using a specific data feature x in range c(x)ϵ[0,10), cϵR), whereas \vec{F} is a vector of binary values in the order of the features (similar to the feature vector), where the added features are listed as 1 and not used features are listed as 0. b represents the index of the batch used during the exploration of the feature node dependency graph and n is the number of all possible features and m is the number of data points in the selected batch b. The following can be configured as an optional design choice

$${t}_{lab} + {t}_{gen} = 0$$

as 0 in the case that the generative model is not needed to be updated. In that case, only time for training and prediction of the end classifier (i.e. the discriminative classifier model) would be considered.

According to an embodiment, the optimization may start with selection of a feature that have the minimum cost in the feature vector (arbitrary selection between possible multiple features as such). The representing node is added to a queue as the current set of features used. An initial batch (i=1) is fed to the system and the initial iteration of the loss function is made. The expected loss L for (i=1) is computed and saved for the next iteration. In the next iteration, an updated set of nodes is explored based on the node dependencies, where independent nodes are favored to be included. Several heuristics can be considered for node inclusion, such as a greedy approach of choosing the most distant node from the existing set, which is not include before. The new loss L is computed and if the loss value is lower than the previous case, and based on that node can be added or not added to the queue. The graph traversal is stochastic and the algorithm includes dropouts in order to avoid local minima and converge to the global minimum for the loss function. For the iterations of the optimization, a set of “training” batches can be re-used where some iterations may share the same “training batches”. Although it is provided a simplistic way in this embodiment as an example for the convergence of the optimization, more advanced methods and optimization tools can be leveraged for making sure that the optimization would converge efficiently to a global minimum.

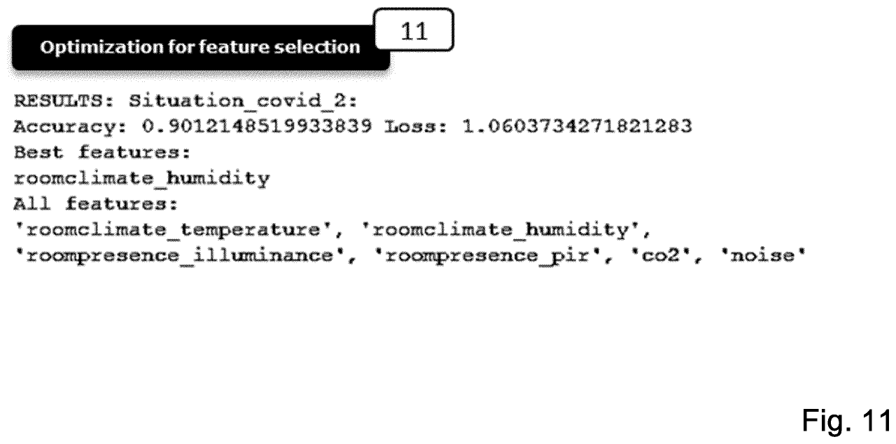

FIG. 11 shows a listing illustrating an optimization for feature selection for a method and a system in accordance with an embodiment of the present invention.

The following algorithm pseudocode provides an example way of optimizing the graph traversal through stochastic inclusion and dropouts of feature nodes:

Algorithm 1 for optimization of the feature node selection

Given graph G, loss function L, feature cost vector \vec{c}, expected batch size
B, inclusion threshold ε₁, loss threshold ε₂

| | |
|---|---|
| Q = [ ] | #feature node queue |
| p=0 | #data point pointer |

while L is not converged (|L_{current} − Lₚᵣₑᵥ | >ε₂)

| | |
|---|---|
| b:= {x_{p ,}xₚ₊₁,...,x_{p+B} } | #data points in the new batch |
| Rand p | #Inclusion or dropout probability |

if (p ≤ ε₁)

| | |
|---|---|
| Qₜₑₘₚ := Q + [nᵢ ]; | #Stochastically include n_{i,} |

else:

| | |
|---|---|
| Qₜₑₘₚ := Q − [nⱼ] | #Stochastic dropout of n_{j ,} |

endif

| | |
|---|---|
| Compute L_{b}(x_{p ,}xₚ₊₁,...,x_{p+B}) | #Given the equation for loss |
| | function |

```
    If L_{b} (x_{p ,}xₚ₊₁,...,x_{p+B-1}) ≤ − μ + L_{b} (x_{p-B ,}x_{p+1-B},...,xₚ₋₁)
      Q := Qₜₑₘₚ
    endif
    p:= p + B
  endwhile
```

| | |
|---|---|
| output Q | # feature node selection |

A system in accordance with an embodiment of the present invention may include one or more of the following components:
1) Mobile devices with sensors carried by people
   Internal software for anonymizing and transmitting sensory data
   Possible sensors: Acoustic sensors, WiFi/BT scanners, infrared sensors, gyroscope, accelerometer, barometer
2) IoT devices deployed in the physical environment
   Internal software for anonymizing and transmitting sensory data
   Raspberry PIs/Arduino devices with built-in or external sensors
      i. Possible sensors: Temperature, humidity, acoustic, WiFi/BT, illuminance Cameras
      i. Possible type of cameras: Stereoscopic cameras, RGB cameras, fisheye cameras
3) Cloud/Edge server(s) with a DB containing the collected data
   Machine learning/pattern recognition software implementing the proposed method
      i. Data programming with labelling functions for predefined situations
      ii. Feature selection algorithm
      iii. Interface for defining labelling functions, labelling function thresholds, feature names, and cost vectors

The above listing includes many possible components for a system in accordance with an embodiment of the invention, whereas not all of the listed sensors or modules would be necessary. Furthermore, the set of available devices may change from time to time and the system may be able to adapt to these changes dynamically. For example, basic requirements might include a server to run pattern recognition (e.g., machine learning) modules, a set of mobile and/or IoT devices with a set of sensors and communication between the devices and the server.

For the usage of cameras and image/video data collection, off-the-shelf anonymization techniques may be considered (e.g., blurring the faces of people, removing the frames with faces). The face detection can be done on the device-side before sending the collected data to the server. Facial recognition on the device- or server-side would not improve the performance of the proposed system.

Embodiments of the invention may assume that usage of each type of sensor might lead to deployment and operations costs. The operation costs may involve energy consumption.

Furthermore, embodiments of the invention may assume that domain knowledge can be obtained from people who have knowledge about the transmission possibilities of diseases in the indoor environments and this knowledge is gathered in the knowledge base. This assumption might lead to inaccuracy for specific cases or environments, where the domain experts do not have any previous knowledge. In those cases, the system according to an embodiment may provide warnings based on overall disease transmission knowledge (e.g., distance between people, duration of time spent with people).

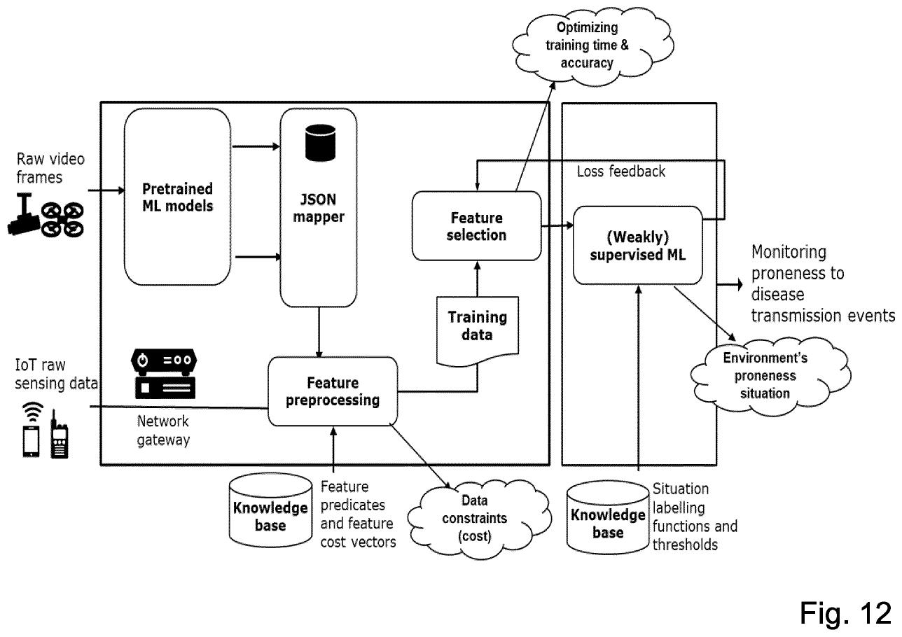

FIG. 12 shows an implementation of a system for monitoring a physical environment's proneness to infectious disease transmission in accordance with an embodiment of the present invention. The system is considered to be implemented using various sensors including IoT sensors (e.g., wireless sensors) and cameras. FIG. 12 illustrates an example implementation (instantiation) of the proposed system. For creating high-level features, various pre-trained machine learning models can be leveraged. These pre-trained models can extract features from image/video frames (e.g., Yolo, OpenPose), raw text data (e.g., extracting text features using WordNet), or others. The features coming from the pre-trained machine learning methods can be combined through a mapper (e.g., JSON mapper), which are then preprocessed along with the other raw data features that are not fed to the pre-trained machine learning models. The knowledge base inputs are given at the preprocessing phase as well as in the classification phase (cf. right side of FIG. 12). The feature selection module runs Algorithm 1 as described above. The classification model is based on the weak supervision approach, which also helps selecting feature nodes through the loss feedback mechanism. The outputs of the system can enable monitoring different environments' proneness to disease transmissions. For instance, proneness of airports, hospitals, schools, offices, and other indoor environments can be monitored for COVID-19 disease transmission.

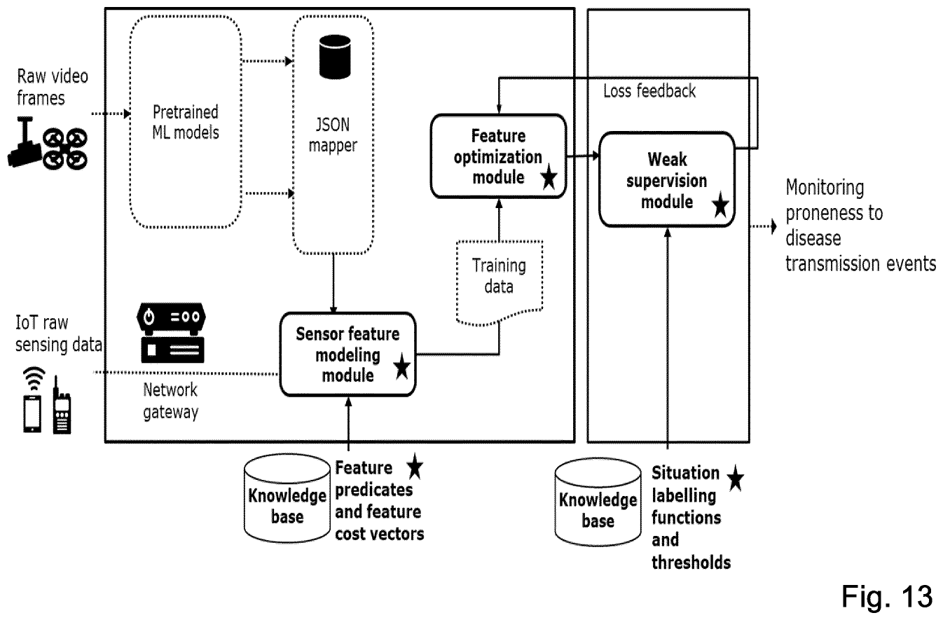

FIG. 13 shows a further implementation of a system for monitoring a physical environment's proneness to infectious disease transmission in accordance with an embodiment of the present invention. Specifically, FIG. 13 shows newly proposed modules unique to this embodiment of a system for disease transmission monitoring. The new modules/components are marked with a star sign.

FIG. 13 marks the components that are uniquely considered in this embodiment for the disease transmission monitoring. The new physical components of the system are as follows:
1) Sensor feature modeling module,
2) Feature optimization module, and
3) Weak supervision module.

The Sensor feature modeling module models the sensor data features based on the inputs received from the knowledge base in a unique way. The feature optimization module optimizes the feature learning based on the unique objective function and graph traversal mechanism. The weak supervision module enables disease monitoring situation labels and uses the knowledge base to set thresholds for these situations. Lastly, the weak supervision module enables the optimization of the feature learning objective in a unique underlying logic as described in accordance with embodiments of the present invention.

### Possible Use Cases:

Embodiments of the invention may be considered for several different situations that can be easily that may lead to environments' proneness to disease transmissions. The collection of video data may enable easy labelling of the described situations by writing heuristic functions and running the function over the datasets that contain streams of sensory data. Such data along with pre-trained machine learning models (e.g., Yolo, MobiNet) can be used as weak supervision data sources for data programming.

Some of the situations that may be monitored for possible disease transmissions are described below. These situations mostly occur in social setups in indoor environments. Thus, some example use cases are as follows:
1. Room environmental dynamics: It is a known fact that various factors such as humidity, room temperature and ventilation change the spread probability of certain diseases such as COVID-19. Various groups work on airborne transmission of such diseases through simulating room environments. Room sensors such as humidity sensor, thermometers, and CO2 sensors can be leveraged for monitoring environmental dynamics for the rooms.
2. Social distances: Certain environments are prone to violation of social distancing even when people give attention to avoid such violations. For instance, when you have a more than allowed number of people mistakenly allowed in an indoor hall, it would be very hard to keep social distancing measures in order. Sensors such as stereoscopic cameras, UWB, WiFi, BT as well as acoustic sensors can be considered for social distance monitoring.
3. Long conversations and social interactions: A system in accordance with an embodiment of the invention may be used for monitoring occurrence of long conversations through various sensors such as cameras and acoustic sensors (e.g., noise in the environment). Long conversations may be a result of the environment's needs. Environments that lead to longer interactions might be considered for changing their setups.
4. Contact situations: A system in accordance with an embodiment of the invention may be considered for monitoring possible contacts to the areas that should have limited access as well as human-to-human contacts. People-centric sensors such as accelerometer, gyroscope, BT, barometer, infrared as well as various room sensors can be leveraged for monitoring these situations.

Other than the above listed examples that may lead to proneness for disease transmissions, other metrics such as movement frequencies of people can be monitored, too.

All of the listed use cases may be implemented without the existence of the ground-truth data. The optimization feedbacks can be received through labelling function or for a simpler case, pre-trained machine learning models (e.g., Yolo or OpenPose) for image processing can be leveraged to provide these situations with high accuracy, while using possible privacy-sensitive data. After the optimization, there would be no real need for collecting image/video data.

Many modifications and other embodiments of the invention set forth herein will come to mind to the one skilled in the art to which the invention pertains having the benefit of the teachings presented in the foregoing description and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

1. A method for monitoring of a proneness of a physical environment to infectious disease transmission, the method comprising:
in a training phase:
obtaining unlabeled sensor data from sensors of the physical environment in order to provide a set of sensor features;
generating, by applying situation labeling functions, a labeling matrix that is fed to a generative model, wherein the generative model feeds a discriminative classifier model with probabilistic labels for the sensor features, wherein the probabilistic labels of the generative model are used for training the discriminative classifier model;
determining, by a feature selection optimizer entity, a subset of the sensor features based on an optimization procedure; and
in an operational phase:
using, by the discriminative classifier model, the subset of sensor features for detecting predefined situations, which make the physical environment prone to infectious disease transmission.

2. The method according to claim 1, further comprising in the training phase:
receiving feature cost information for the sensor features from a knowledge base, wherein the feature cost information is used for considering a predetermined constraint metric for the physical environment.

3. The method according to claim 1, further comprising in the training phase:
receiving sensor feature predicates from a knowledge base, wherein the sensor feature predicates specify characteristics of the sensor features.

4. The method according to claim 3, wherein a match score is employed for considering a level of matches of sensor feature predicates between two sensor features.

5. The method according to claim 3, further comprising in the training phase:
generating a feature node dependency graph based on the sensor feature predicates of the sensor features, wherein a match between the sensor feature predicates of two sensor features constitutes a dependency between the two sensor features.

6. The method according to claim 5, wherein the feature node dependency graph is generated in such a way that the sensor features are represented by vertices, connections between sensor features are represented by edges, and match scores are represented by edge weights.

7. The method according to claim 5, wherein the optimization procedure of the feature selection optimizer entity is based on a traversal of the feature node dependency graph.

8. The method according to claim 5, wherein the optimization procedure includes an optimization function, and wherein the optimization function is built based on the feature node dependency graph based on the edge weight values.

9. The method according to claim 1, wherein the optimization procedure includes an optimization function, wherein the optimization function is built based on a feature cost vector, wherein the feature cost vector includes feature cost information for the sensor features.

10. The method according to claim 1, wherein the optimization procedure includes an optimization function, wherein the optimization function is built based on training and/or prediction times of the generative model and/or of the discriminative classifier model.

11. The method according to claim 1, wherein the optimization procedure includes an optimization function, wherein the optimization function is built based on prediction accuracy and/or confidence values of the discriminative classifier model.

12. The method according to claim 1, wherein, in the training phase, the feature selection optimizer entity iteratively interacts with the discriminative classifier model such that the subset of sensor features is iteratively updated based on feedback information that is provided by the discriminative classifier model.

13. The method according to claim 1, wherein the probabilistic labels of the generative model are based on the unlabeled sensor data and predetermined situation labeling thresholds.

14. A system for monitoring of a proneness of a physical environment to infectious disease transmission, the system comprising a functional unit having one or more computational processors with access to memory, which, alone or in combination, are configured to provide for execution of the following steps:
in a training phase:
obtaining unlabeled sensor data from sensors of the physical environment in order to provide a set of sensor features;
generating, by applying situation labeling functions, a labeling matrix that is fed to a generative model, wherein the generative model feeds a discriminative classifier model with probabilistic labels for the sensor features, wherein the probabilistic labels of the generative model are used for training the discriminative classifier model;
determining, by a feature selection optimizer entity, a subset of the sensor features based on an optimization function; and
in an operational phase:
using, by the discriminative classifier model, the subset of sensor features for detecting predefined situations, which make the physical environment prone to infectious disease transmission.

15. A non-transitory, computer-readable storage medium having instructions thereon, which, upon execution on one or more processors, provide for execution of the following steps:
in a training phase:
obtaining unlabeled sensor data from sensors of the physical environment in order to provide a set of sensor features;
generating, by applying situation labeling functions, a labeling matrix that is fed to a generative model, wherein the generative model feeds a discriminative classifier model with probabilistic labels for the sensor features, wherein the probabilistic labels of the generative model are used for training the discriminative classifier model;
determining, by a feature selection optimizer entity, a subset of the sensor features based on an optimization function; and
in an operational phase:
using, by the discriminative classifier model, the subset of sensor features for detecting predefined situations, which make the physical environment prone to infectious disease transmission.
